(19) United States
(12) Patent Application Publication
(10) Pub. No.: US 2025/0160710 A1
(43) Pub. Date: May 22, 2025

(54) Computer System and Emotion Estimation Method

(71) Applicant: Hitachi, Ltd., Chiyoda-ku, Tokyo (JP)

(72) Inventors: Yoshihisa MATSUNAGA, Tokyo (JP); Zisheng LI, Tokyo (JP); Masahiro OGINO, Tokyo (JP); Peifei ZHU, Tokyo (JP)

(21) Appl. No.: 18/689,540

(22) PCT Filed: Aug. 12, 2022
(86) PCT No.: PCT/JP2022/030813

§ 371 Date: Dec. 26, 2024

(30) Foreign Application Priority Data
Dec. 16, 2021 (JP) 2021-204416

(51) Int. Cl. A61B 5/16; A61B 5/024; A61B 5/11

(52) U.S. Cl. CPC A61B 5/165; A61B 5/024; A61B 5/11

(57) ABSTRACT

A computer system is configured to: obtain, from a user, biological data including a biological signal of the user and motion data including a motion signal relating to a motion of the user and store the biological data and the motion data in the storage device; generate a biological signal time series and a motion signal time series through use of the biological data and the motion data in any time range; correct, through use of the motion signal time series, the biological signal time series to a corrected biological signal time series having reduced influence of the motion of the user; and estimate emotion of the user through use of a first biological feature amount calculated from the corrected biological signal time series, and store an emotion estimation result of the user in the storage device.

### INCORPORATION BY REFERENCE

This application claims priority to Japanese Patent Application No. 2021-204416 filed on Dec. 16, 2021, the content of which is incorporated herein by reference.

### BACKGROUND OF THE INVENTION

This disclosure relates to a system and method for estimating emotion.

In recent years, in the field of health care, there has been a progress in an attempt to feed back emotion of a user to provide and promote development of a better service. In the development of the technology of reflecting the emotion of the user in order to increase the quality of life (QoL), there is a progress in use of an emotion estimation technology that uses a biological signal which can be obtained from a biological sensor for monitoring a state of the user during a living activity.

For example, as the background art of this disclosure, a technology as described in JP 2017-121286 A is known. In JP 2017-121286 A, it is disclosed that “An emotion estimation system includes an acquiring unit 131, a classifying unit, and an output control unit 137. The acquiring unit acquires information on one user's heartbeat intervals measured continuously. The classifying unit classifies user's emotion as any one of at least two types of emotion based on the basis of a value indicating a ratio of a value obtained as a result of frequency analysis of the acquired heartbeat interval information to a value indicating a gap between a predicted heartbeat interval calculated on the basis of the acquired heartbeat interval information and an actually obtained heartbeat interval. The output control unit performs a different output according to a result of the classifying.”

### SUMMARY OF THE INVENTION

In JP 2017-121286 A, there is described a system which performs output according to an emotional abnormal state. However, the technology as described in JP 2017-121286 A is an estimation method for emotion in a mental state relating to stress, and hence estimates positive emotion as a relaxed state. Thus, this technology cannot estimate detailed emotion. Moreover, the technology as described in JP 2017-121286 A uses only heart rate variability, and does not consider influence of an action of the subject on the emotion estimation.

This invention has an object to provide a system and a method with which influence of an action of a person on emotion estimation is reduced, to thereby highly accurately estimate various types of emotion.

A representative example of the present invention disclosed in this specification is as follows: a computer system comprises at least one computer including a processor, a storage device coupled to the processor, and a coupling interface coupled to the processor. The processor is configured to: obtain, from a user, biological data including a biological signal of the user and motion data including a motion signal relating to a motion of the user and store the biological data and the motion data in the storage device; generate a biological signal time series and a motion signal time series through use of the biological data and the motion data in any time range; correct, through use of the motion signal time series, the biological signal time series to a corrected biological signal time series having reduced influence of the motion of the user; and estimate emotion of the user through use of a first biological feature amount calculated from the corrected biological signal time series, and store an emotion estimation result of the user in the storage device.

According to the one aspect of this disclosure, the emotion of a person can highly accurately be estimated. Problems, configurations, and effects other than those described above become apparent from the following description of an embodiment.

### BRIEF DESCRIPTION OF THE DRAWINGS

The present invention can be appreciated by the description which follows in conjunction with the following figures, wherein:

FIG. 1 is a diagram for illustrating a configuration example of a system according to a first embodiment of this invention;

FIG. 2 is a diagram for illustrating an example of a hardware configuration of a computer included in an emotion estimation system in the first embodiment;

FIG. 3 is a table for showing an example of a data structure of data stored in user data management DB in the first embodiment;

FIG. 4 is a table for showing an example of a data structure of data stored in emotion estimation result management DB in the first embodiment;

FIG. 5 is a table for showing an example of a data structure of data stored in learning data management DB in the first embodiment;

FIG. 6 is a diagram for illustrating a flow of emotion estimation processing executed by the emotion estimation system in the first embodiment;

FIG. 7 is a flowchart for illustrating an example of the emotion estimation processing executed by the emotion estimation system in the first embodiment;

FIG. 8 is a graph for showing an example of a heartbeat time series in the first embodiment;

FIG. 9 is a graph for showing an example of an acceleration time series in the first embodiment;

FIG. 10 is a graph for showing an example of a frequency spectrum of the heartbeat time series in the first embodiment;

FIG. 11 is a flowchart for illustrating an example of learning processing for an emotion estimation model executed by the emotion estimation system in the first embodiment;

FIG. 12 is a diagram for illustrating an example of an emotion model applied to the emotion estimation system in the first embodiment;

FIG. 13 is a diagram for illustrating a flow of learning processing for a correction model executed by the emotion estimation system in the first embodiment;

FIG. 14 is a flowchart for illustrating an example of the learning processing for the correction model executed by the emotion estimation system in the first embodiment;

FIG. 15 is a diagram for illustrating a flow of learning processing for the correction model executed by the emotion estimation system in the first embodiment; and

FIG. 16 is a view for illustrating an example of a screen presented by the emotion estimation system in the first embodiment.

### DETAILED DESCRIPTION OF THE PREFERRED EMBODIMENTS

Now, description is given of at least one embodiment of this invention referring to the drawings. It should be noted that this invention is not to be construed by limiting the invention to the content described in the following at least one embodiment. A person skilled in the art would easily recognize that specific configurations described in the following at least one embodiment may be changed within the scope of the concept and the gist of this invention.

In configurations of the at least one embodiment of this invention described below, the same or similar components or functions are denoted by the same reference numerals, and a redundant description thereof is omitted here.

Notations of, for example, “first,” “second,” and “third” herein are assigned to distinguish between components, and do not necessarily limit the number or order of those components.

The position, size, shape, range, and others of each component illustrated in, for example, the drawings may not represent the actual position, size, shape, range, and others in order to facilitate understanding of this invention. Thus, this invention is not limited to the position, size, shape, range, and others disclosed in, for example, the drawings.

### First Embodiment

An emotion estimation system is now described. The emotion estimation system evaluates, for example, a QoL in a mental region in the health care field. It is possible to achieve provision and development of a service reflecting emotion of a person in a daily life by using an evaluation result. An emotion estimation method according to this disclosure can be applied without a limit imposed by personality and an environment of a user.

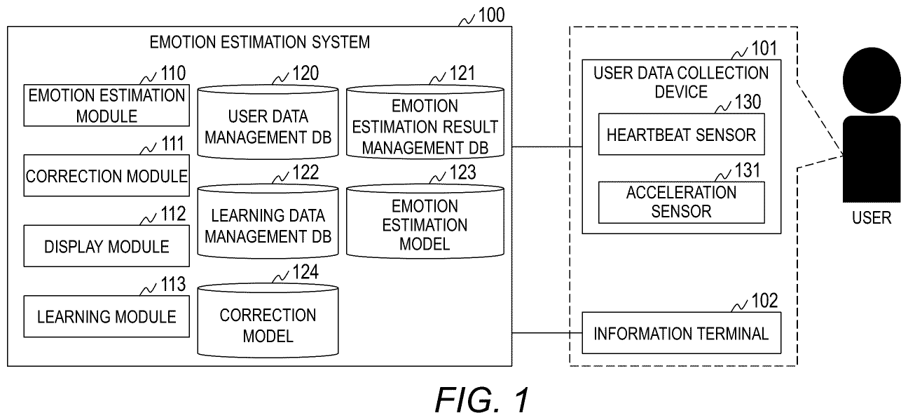

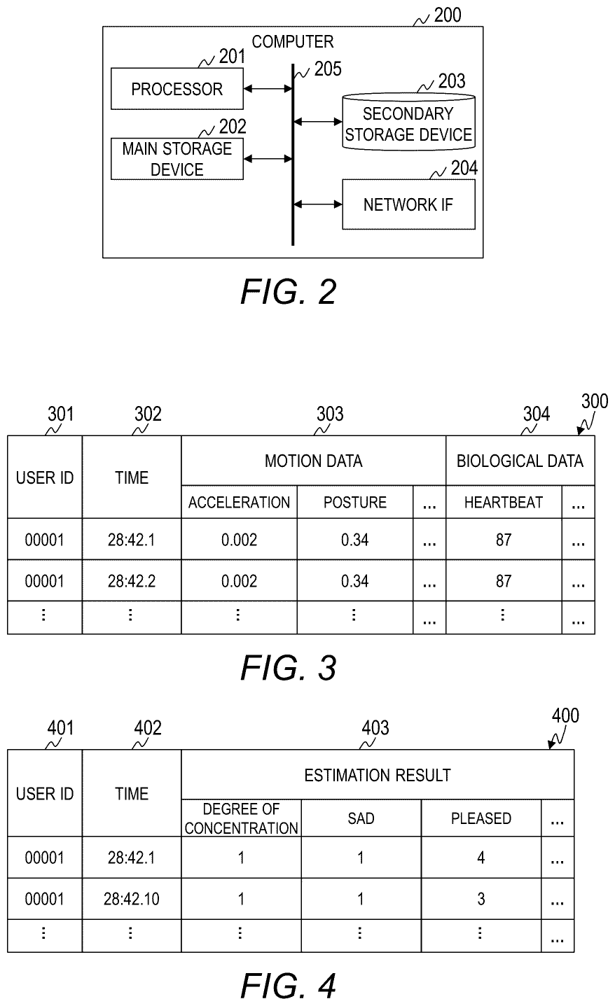

FIG. 1 is a diagram for illustrating a configuration example of a system according to a first embodiment of this invention. FIG. 2 is a diagram for illustrating an example of a hardware configuration of a computer included in an emotion estimation system in the first embodiment.

The system includes an emotion estimation system 100, a user data collection device 101, and an information terminal 102. The user data collection device 101 and the information terminal 102 are coupled to the emotion estimation system 100 via a network. The network is, for example, a local area network (LAN) or the like, and may be of any one of a wired type or a wireless type.

The user data collection device 101 is a device worn by a user, and includes biological data measurement devices such as a heartbeat sensor 130 and motion data measurement devices such as an acceleration sensor 131. The user data collection device 101 may include, as the biological data measurement devices, sensors which measure a brain wave, a skin potential, a body temperature, and the like, and may include a gyroscope as the motion data measurement device. Moreover, the user data collection device 101 may include a measurement device which measures a value relating to the environment of the user, such as an atmospheric pressure sensor. On the user data collection device 101, various sensors can be installed as long as the sensors do not obstruct the daily life of the user.

The user data collection device 101 obtains measurement results from the biological data measurement devices and the motion data measurement devices, generates user data including biological data and motion data, and transmits the user data to the emotion estimation system 100.

The information terminal 102 is a terminal to be operated by the user, such as a smartphone or a personal computer. The emotion estimation system 100 transmits, to the information terminal 102, state information on the user including an estimation result of the emotion, the biological data, and the motion data. Details of a screen displayed on the information terminal 102 are described later.

The emotion estimation system 100 estimates the emotion, and executes learning of various models used for the estimation of the emotion. The emotion estimation system 100 is formed of a computer 200 as illustrated in FIG. 2. The number of computers 200 which form the emotion estimation system 100 may be one, or two or more.

The computer 200 includes a processor 201, a main storage device 202, a secondary storage device 203, and a network interface 204. The hardware elements are coupled to each other through an internal bus 205.

The processor 201 executes a program stored in the main storage device 202. The processor 201 executes processing in accordance with the program, to thereby operate as a function module (module) for implementing a specific function. In the following description, when the processing is described with a function module as the subject, the description indicates that the processor 201 is executing the program for implementing the function module.

The main storage device 202 is a dynamic random access memory (DRAM), and stores programs to be executed by the processor 201 and data to be used by the programs. The main storage device 202 is also used as a work area.

The secondary storage device 203 is a hard disk drive (HDD), a solid state drive (SSD), or the like, and permanently stores data. The programs and data stored in the main storage device 202 may be stored in the secondary storage device 203. In this case, the processor 201 reads out the programs and information from the secondary storage device 203, and loads the programs and information onto the main storage device 202.

The network interface 204 is an interface for coupling to an external device through a network.

Such a hardware configuration of the computer 200 is merely an example, and this invention is not limited thereto. The computer 200 may include input devices such as a keyboard, a mouse, and a touch panel, or may include output devices such as a display and a printer.

The emotion estimation system 100 includes, as function modules, an emotion estimation module 110, a correction module 111, a display module 112, and a learning module 113. Moreover, the emotion estimation system 100 holds a user data management DB 120, an emotion estimation result management DB 121, a learning data management DB 122, an emotion estimation model 123, and a correction model 124.

The emotion estimation model 123 is a model for estimating the emotion of a person. The emotion estimation model 123 is a model for estimating the emotion through use of the biological data on the user in a state close to a calm state, and is, for example, a regression model or a neural network. The state close to the calm state indicates a state in which the user does not move much and is settled. The correction model 124 is a model generated from the biological data, and is a model for correcting a time series used for the emotion estimation. The correction model 124 is, for example, a regression model or a neural network.

The user data management DB 120 is a database which stores the user data received from the user data collection device 101. The emotion estimation result management DB 121 is a database which stores a result of the emotion estimation. The learning data management DB 122 is a database which stores learning data used for learning of the emotion estimation model 123 and the correction model 124.

The correction module 111 corrects the time series through use of the motion data and the correction model 124. In this embodiment, the time series is corrected to a time series having reduced influence of the motion. The emotion estimation module 110 estimates the emotion of the person through use of the corrected time series and the emotion estimation model 123. Specifically, the emotion estimation module 110 estimates a type and a level of the emotion in any emotion model, and outputs the estimation result of the emotion. The display module 112 presents the estimation result of the emotion, the state information on the user, and the like. The learning module 113 executes the learning of the emotion estimation model 123 and the correction model 124.

Regarding the respective function modules of the emotion estimation system 100, a plurality of function modules may be combined into one function module, or one function module may be divided into a plurality of function modules each corresponding to a relevant function. For example, the learning module 113 may be divided into a learning module which executes the learning of the emotion estimation model 123 and a learning module which executes the learning of the correction model 124. Moreover, the emotion estimation module 110 may include the correction module 111.

FIG. 3 is a table for showing an example of a data structure of the data stored in the user data management DB 120 in the first embodiment.

In the user data management DB 120, a table 300 as shown in FIG. 3 is stored. The table 300 stores entries each including a user ID 301, a time 302, motion data 303, and biological data 304. One entry corresponds to one piece of user data. It should be noted that the fields contained in the entry are not limited to those described above. Any one of the above-mentioned fields may not be included, or another field may be included.

The user ID 301 is a field which stores identification information on the user. The time 302 is a field which stores a time at which the user data was generated. The motion data 303 is a field group which stores values measured by the motion data measurement devices. In FIG. 3, values relating to an acceleration and a posture are stored. The biological data 304 is a field group which stores values measured by the biological data measurement devices. In FIG. 3, a value relating to the heartbeat is stored.

FIG. 4 is a table for showing an example of a data structure of the data stored in the emotion estimation result management DB 121 in the first embodiment.

In the emotion estimation result management DB 121, a table 400 as shown in FIG. 4 is stored. The table 400 stores entries each including a user ID 401, a time 402, and an estimation result 403. One entry corresponds to one emotion estimation result. It should be noted that the fields contained in the entry are not limited to those described above. Any one of the above-mentioned fields may not be included, or another field may be included.

The user ID 401 is the same field as the user ID 301. The time 402 is a field which stores a start time of the time series of the user data used to execute the emotion estimation. In this embodiment, a time series of the user data having a predetermined time width is used to estimate the emotion. The estimation result 403 is a field group which stores values output from the emotion estimation module 110. In the estimation result 403 of FIG. 4, a degree of concentration and a degree of each emotion type are stored.

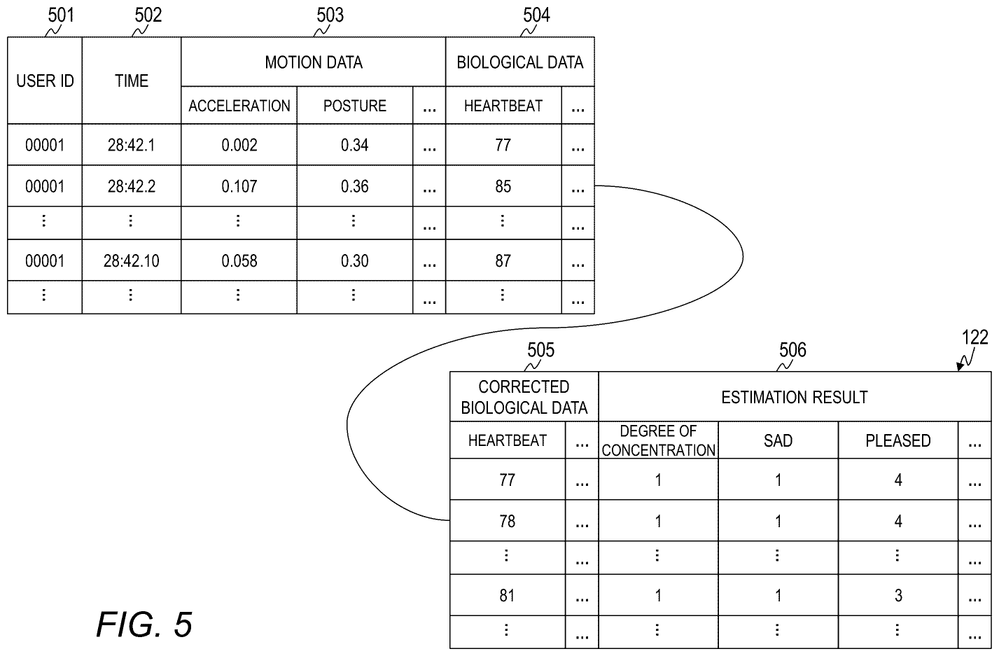

FIG. 5 is a table for showing an example of a data structure of the data stored in the learning data management DB 122 in the first embodiment.

In the learning data management DB 122, a table 500 as shown in FIG. 5 is stored. The table 500 is a table which stores the learning data used to execute learning of the emotion estimation model 123, and stores entries each including a user ID 501, a time 502, motion data 503, biological data 504, corrected biological data 505, and an estimation result 506. One entry corresponds to one piece of user data. It should be noted that the fields contained in the entry are not limited to those described above. Any one of the above-mentioned fields may not be included, or another field may be included.

The user ID 501, the time 502, the motion data 503, and the biological data 504 are the same fields as the user ID 301, the time 302, the motion data 303, and the biological data 304, respectively. The corrected biological data 505 is a field group which stores corrected biological data. The estimation result 506 is a field group which stores the estimation results of the emotion estimated through use of the time series of the user data. The same values are set to the estimation result 506 of an entry corresponding to the user data forming the time series used to output one estimation result.

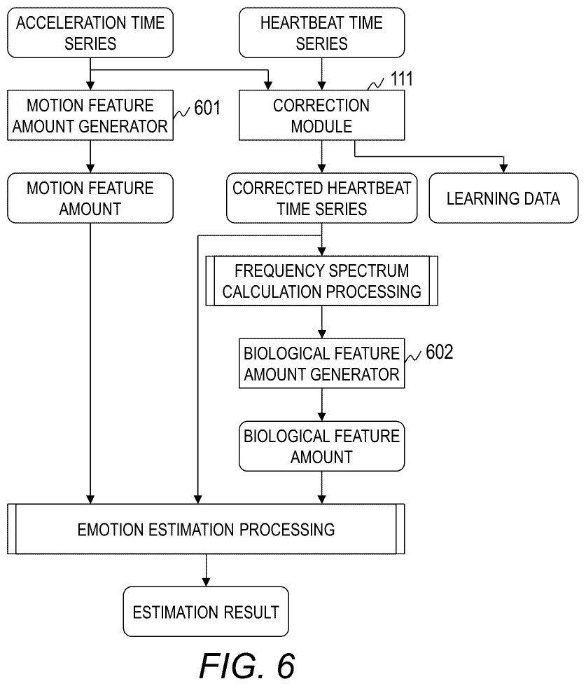

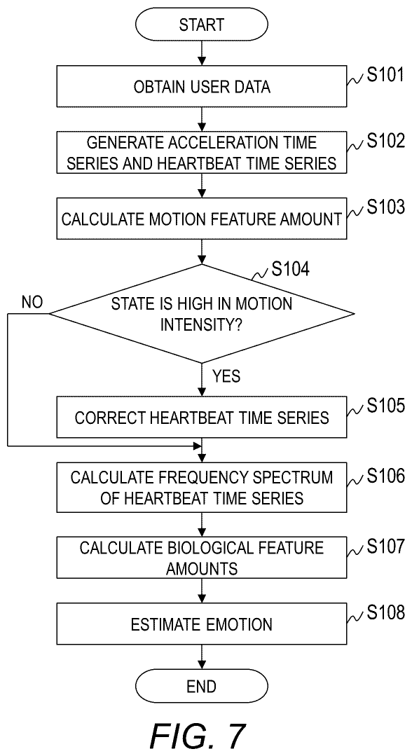

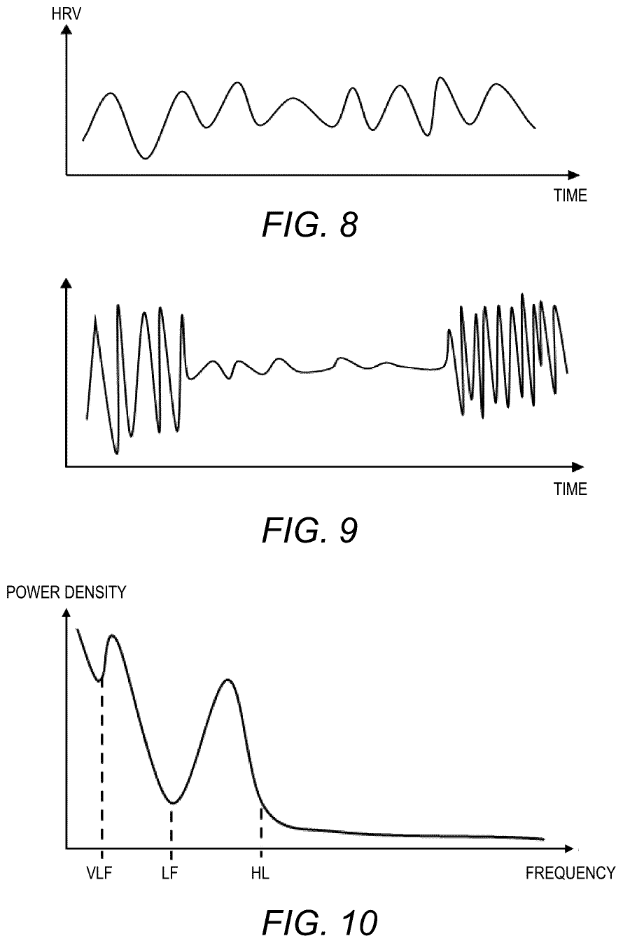

An emotion estimation method according to the first embodiment is now described. FIG. 6 is a diagram for illustrating a flow of emotion estimation processing executed by the emotion estimation system 100 in the first embodiment. FIG. 7 is a flowchart for illustrating an example of the emotion estimation processing executed by the emotion estimation system 100 in the first embodiment. FIG. 8 is a graph for showing an example of a heartbeat time series in the first embodiment. FIG. 9 is a graph for showing an example of an acceleration time series in the first embodiment. FIG. 10 is a graph for showing an example of a frequency spectrum of the heartbeat time series in the first embodiment.

In the following description, description is given of a method of estimating the emotion through use of the acceleration included in the motion data and the heartbeat included in the biological data.

The emotion estimation module 110 obtains the user data having the predetermined time width (Step S101).

The emotion estimation module 110 generates the time series relating to the acceleration (acceleration time series) and the time series relating to the heartbeat (heartbeat time series) based on a plurality of pieces of obtained user data (Step S102). FIG. 8 shows the example of the heartbeat time series. The horizontal axis represents the time, and the vertical axis represents an HRV being the number of heartbeats per unit time being a millisecond. FIG. 9 shows the example of the acceleration time series. The horizontal axis represents the time, and the vertical axis represents the acceleration.

The emotion estimation module 110 inputs the acceleration time series to a motion feature amount generator 601, to thereby calculate a motion feature amount (Step S103). The motion feature amount generator 601 calculates, for example, a frequency of alternation between positive and negative of the acceleration (zero-crossing amount) as the motion feature amount.

The emotion estimation module 110 determines whether or not the state is high in motion intensity based on the motion feature amount (Step S104). For example, in a case where the motion feature amount is higher than a threshold value, the emotion estimation module 110 determines that the state is high in motion intensity. In a case where the state is not high in motion intensity, the emotion estimation module 110 advances the process to Step S106. In a case where the state is higher in motion intensity than a certain degree, the heartbeat time series is highly possibly influenced by the motion, and hence correction of reducing the influence of the motion is made.

In a case where the state is high in motion intensity, the emotion estimation module 110 invokes the correction module 111 and instructs the correction module 111 to correct the heartbeat time series. At this time, the emotion estimation module 110 outputs the motion feature amount and the heartbeat time series to the correction module 111. The correction module 111 uses the motion feature amount and the heartbeat time series, and the correction model 124 to correct the heartbeat time series (Step S105). As a result of this correction, the heartbeat time series is corrected to a heartbeat time series having reduced influence of the motion. In other words, the heartbeat time series is corrected to a heartbeat time series in a state close to a calm state. The correction module 111 outputs the corrected heartbeat time series to the emotion estimation module 110. In a case where the emotion estimation module 110 receives the corrected heartbeat time series, the emotion estimation module 110 advances the process to Step S106.

In Step S106, the emotion estimation module 110 uses the heartbeat time series to calculate the frequency spectrum of the heartbeat time series (Step S106).

FIG. 10 is a graph for showing the example of the frequency spectrum of the HRV. The horizontal axis represents a frequency, and the vertical axis represents a spectrum power density of the HRV.

The emotion estimation module 110 inputs the heartbeat time series and the frequency spectrum of the HRV to a biological feature amount generator 602, to thereby calculate the biological feature amount (Step S107).

The biological feature amount generator 602, for example, uses the frequency spectrum of the HRV to calculate spectral power densities of a very-low frequency VLF, a lower frequency LF, and a high frequency HF, and further uses each spectral power density to calculate a total power, a ratio between the spectral power densities of the LF and the HF, an LF Norm, an HF Norm, and the like as the biological feature amounts. The total power is a sum of the spectral power densities of the VLF, the LF, and the HF. The LF Norm is a value which uses the spectral power densities of the LF and the VLF to enhance a feature of the LF. The HF Norm is a value which uses the spectral power densities of the HF and the VLF to enhance a feature of the HF.

The biological feature amount generator 602, for example, the emotion estimation module 110, uses the heartbeat time series to calculate, as the biological feature amounts, an HR, an SDNN, an RMSSD, an NN 50, a pNN 50, an NN 20, a pNN 20, and the like. The HR is an average value of the heart rate. The SDNN is a standard deviation of the HRV. The RMSSD is a root of a mean square of differences each between the HRVs next to each other in succession. The NN 50 is a total number of differences each of which is between the HRVs next to each other in succession, and exceeds 50 milliseconds. The pNN 50 is a ratio of the heartbeats each having the difference which is between the HRVs next to each other in succession and exceeds 50 milliseconds. The NN 20 is a total number of differences each of which is between the HRVs next to each other in succession, and exceeds 20 milliseconds. The pNN 20 is a ratio of the heartbeats each having the difference which is between the HRVs next to each other in succession and exceeds 20 milliseconds.

The emotion estimation module 110 uses the heartbeat time series, the biological feature amounts, and the motion feature amount and the emotion estimation model 123 to estimate the emotion (Step S108). The emotion estimation module 110 stores the estimation result of the emotion in the emotion estimation result management DB 121. The emotion estimation model 123 may be a model which does not use the motion feature amount and the heartbeat time series.

In this embodiment, the emotion estimation module 110 obtains a parameter of any emotion model from the emotion estimation model 123, and uses the parameter and the emotion model to classify the emotion.

The emotion estimation module 110 stores, in the table 500 of the learning data management DB 122, the learning data obtained by associating the user data, the correction result, and the emotion estimation result with one another.

In a case where the heartbeat time series is not corrected, the heartbeat time series without correction is used to execute the frequency spectrum calculation processing in FIG. 6. Moreover, the time series without correction, the biological feature amounts, and the motion feature amount and the emotion estimation model 123 are used to estimate the emotion.

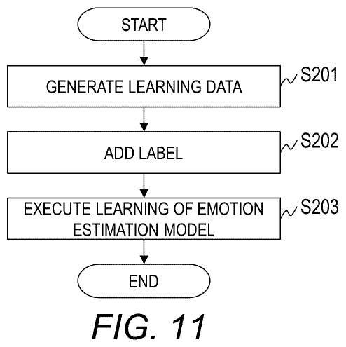

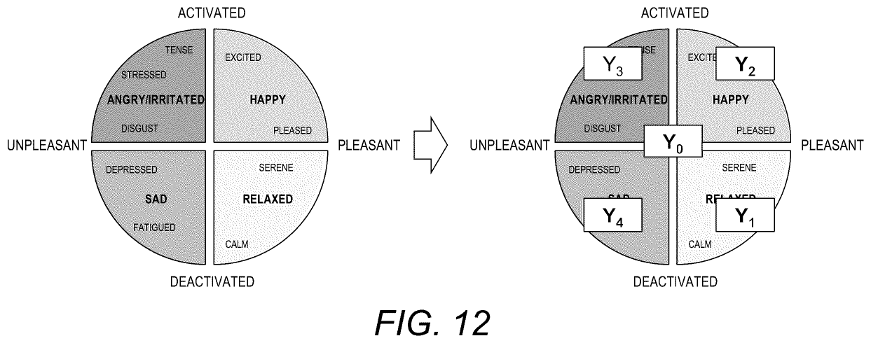

A learning method for the emotion estimation model 123 in the first embodiment is now described. FIG. 11 is a flowchart for illustrating an example of learning processing for the emotion estimation model 123 executed by the emotion estimation system 100 in the first embodiment. FIG. 12 is a diagram for illustrating an example of the emotion model applied to the emotion estimation system 100 in the first embodiment.

In this embodiment, as illustrated in FIG. 12, the emotion model such as the Russell's circumplex model of emotions based on the dimensional theory of emotion or the like is used to estimate the emotion. The circumplex model of emotions illustrated in FIG. 12 has the vertical axis representing arousal and the horizontal axis representing valence. Moreover, in this embodiment, each emotion of the circumplex model of emotions is classified into one of labels (Y₀, Y₁, Y₂, Y₃, and Y₄).

The learning module 113 uses the user data and the emotion estimation result to generate learning data (Step S201).

In this embodiment, the learning module 113 associates the emotion estimation result output through use of the time series in the predetermined time range to one piece of user data, to thereby generate an entry of FIG. 5, and stores the entry in the table 500. The corrected biological data 505 of the generated entry may be blank.

The learning module 113 adds the label in the circumplex model of emotions to the learning data (Step S202). The label added to the learning data represents a ground truth of the estimation result of the emotion estimation model 123.

The learning module 113 uses the learning data to execute learning of the emotion estimation model 123 (Step S203). The learning module 113 stores a learning result as the emotion estimation model 123. As the learning method, it is only required to use a publicly-known method, and hence a detailed description thereof is omitted.

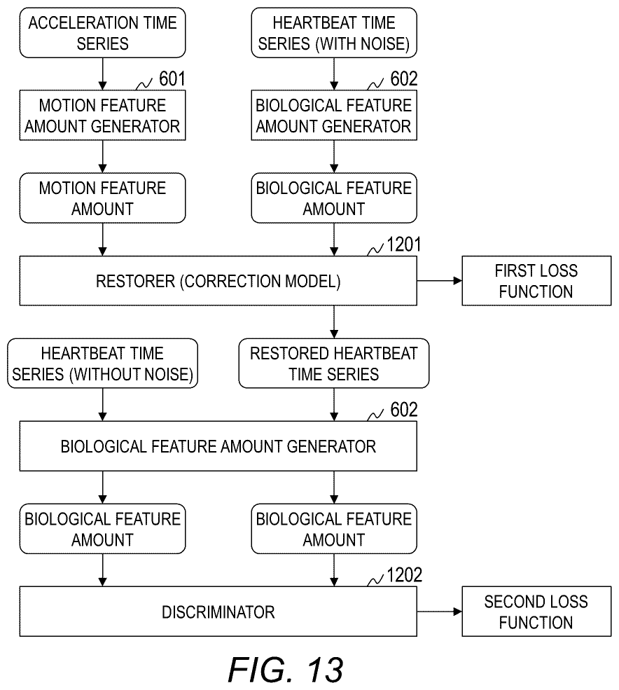

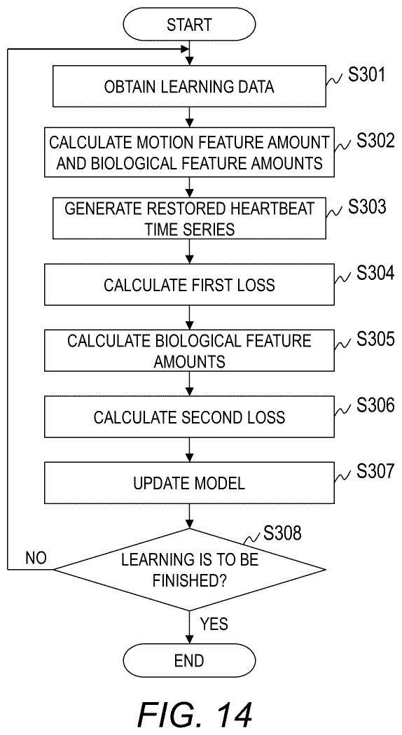

A learning method for the correction model 124 in the first embodiment is now described. FIG. 13 is a diagram for illustrating a flow of learning processing for the correction model 124 executed by the emotion estimation system 100 in the first embodiment. FIG. 14 is a flowchart for illustrating an example of the learning processing for the correction model 124 executed by the emotion estimation system 100 in the first embodiment.

The learning module 113 obtains the learning data (entry group of the table 500) from the learning data management DB 122 (Step S301). The learning data includes the time series of the user data and the corrected heartbeat time series in any time width of any user.

The learning module 113 uses the time series of the user data included in the learning data to calculate the motion feature amount and the biological feature amounts (Step S302). A calculation method for the motion feature amount and the biological feature amounts is the same as that in the emotion estimation.

The learning module 113 inputs the motion feature amount and the biological feature amounts to a restorer 1201, to thereby generate a restored heartbeat time series (Step S303). A model which implements the restorer 1201 is the correction model 124.

The learning module 113 uses a first loss function to calculate a first loss G (Step S304). The first loss function is given by, for example, Equation (1).

Equation (1)

G=β∥y−x∥²−D_{KL}(N_{(μ,σ)}|N_{(0,1)} (1)

As illustrated in FIG. 13, the biological feature amount generator 602 functions as an encoder of an auto-encoder, and the restorer 1201 functions as a decoder of the auto-encoder. The feature amount generated by the encoder is a feature amount which represents the data input to the encoder. It is assumed that the encoder in this embodiment calculates the feature amount through the dimension reduction.

Symbol y represents the restored heartbeat time series generated by the restorer 1201, and symbol x represents the heartbeat time series generated from the user data. D_{KL}(|) represents a Kullback-Leibler divergence, μ represents an average of the biological feature amount, and σ represents a variance of the biological feature amount. N represents a probability distribution.

The learning module 113 inputs the restored heartbeat time series and a heartbeat time series without noise corresponding to the estimated emotion to the biological feature amount generator 602, to thereby calculate the biological feature amounts of each time series (Step S305). The heartbeat time series without noise is a heartbeat time series for certain emotion in the calm state. This heartbeat time series may be set in advance, or may be retrieved from the learning data management DB 122.

The learning module 113 inputs two biological feature amounts to a discriminator 1202, and uses a discrimination result obtained by the discriminator 1202 and a second loss function to calculate a second loss D (Step S306). The discriminator 1202 discriminates whether or not the input heartbeat time series is the heartbeat time series without noise.

As illustrated in FIG. 13, the restorer 1201 and the discriminator 1202 form a generative adversarial network. The learning progresses so that the discriminator 1202 cannot discriminate the restored heartbeat time series generated by the restorer 1201 and the heartbeat time series from each other. The emotion estimation system 100 holds a model which implements the discriminator 1202.

The learning module 113 uses the first loss G and the second loss D to update the model of each of the restorer 1201 and the discriminator 1202 (Step S307).

Specifically, the learning module 113 obtains a solution of a minimization problem given by Equation (2) to update the model. V_{(θ}_{_{G}}_{, θ}_{_{D}}₎ in Equation (2) is given by Equation (3).

$$\begin{matrix}Equation ( 2 ) & \\ {min}_{{θ}_{G}} {min}_{{θ}_{D}} V ( {θ}_{G} , {θ}_{D} ) & ( 2 )\end{matrix}$$

$$\begin{matrix}Equation ( 3 ) & \\ V ( {θ}_{G} , {θ}_{D} ) = {E}_{x ∼ {p}_{t}} [ log {D}_{x} ] + {E}_{z ∼ {p}_{z}} [ 1 - log D ( G ( z ) ) ] & ( 3 )\end{matrix}$$

In Equation (3), θ represents a gradient and E represents binary cross-entropy.

The learning module 113 determines whether or not to finish the learning (Step S307). For example, in a case where the number of times of the update is larger than a threshold value, the learning module 113 determines to finish the learning. Moreover, in a case where a prediction accuracy of the emotion for user data for evaluation is higher than a threshold value, the learning module 113 determines to finish the learning.

In a case where the learning is not to be finished, the learning module 113 returns the process to Step S301, and executes similar processing for new learning data. In a case where the learning is to be finished, the learning module 113 finishes the learning processing for the correction model 124.

As a result of the above-mentioned learning, the correction model 124 which outputs the heartbeat time series having reduced influence of the motion of the user can be obtained. The emotion of the user can highly accurately be estimated without the influence of the motion of the user by using the heartbeat time series corrected by the correction model 124. Moreover, explainability for the determination of the emotion estimation result can be guaranteed by using the biological feature amounts.

According to this embodiment, the emotion of the user can highly accurately be estimated without reorganization or the like of the emotion estimation model 123 which has learned the heartbeat data obtained in the calm state. Thus, such effects as reductions in development cost and operation cost are also provided.

The generative adversarial network used for the learning of the correction model 124 contributes to problem solution of domain adaptation, and hence there exists not only an effect of reducing influence of the motion of the user, but also an effect of reducing influence of the personality and the environment of the user.

The correction of the time series includes processing such as correction of values of the time series and interpolation of the values in the time series.

### Modification Example

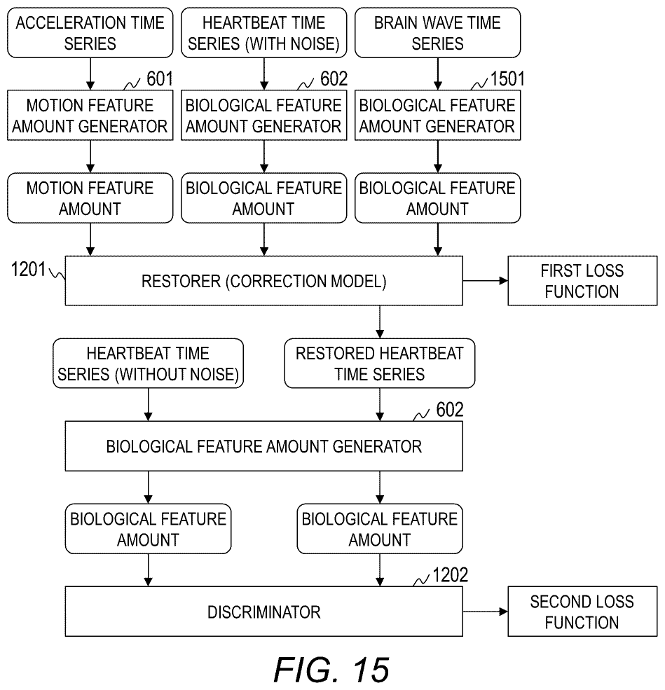

Time series of biological values other than the heartbeat, such as the brain wave, the skin potential, and the body temperature, may be input to the correction model 124. FIG. 15 is a diagram for illustrating a flow of the learning of the correction model 124 which treats the acceleration, the heartbeat, and the brain wave. The correction model 124 is different in such a point that a biological feature amount generator 1501 which generates a biological feature amount relating to the brain wave from a brain wave time series is added. In this case, the first loss function is given by, for example, Equation (4).

$$\begin{matrix}Equation ( 4 ) & \\ G = β {‖ y - x ‖}^{2} - {D}_{K L a} ( {N}_{( μ , σ )} \vert {N}_{( 0 , 1 )} ) - {D}_{K L b} ( {N}_{( μ , σ )} \vert {N}_{( 0 , 1 )} ) & ( 4 )\end{matrix}$$

In this equation, D_{KLa}(|) represents a Kullback-Leibler divergence relating to the acceleration and the heartbeat, and D_{KLb}(|) represents a Kullback-Leibler divergence relating to the brain wave and the heartbeat.

The influence of the motion of the user is possibly reduced more efficiently by using the time series of the biological value other than the heartbeat.

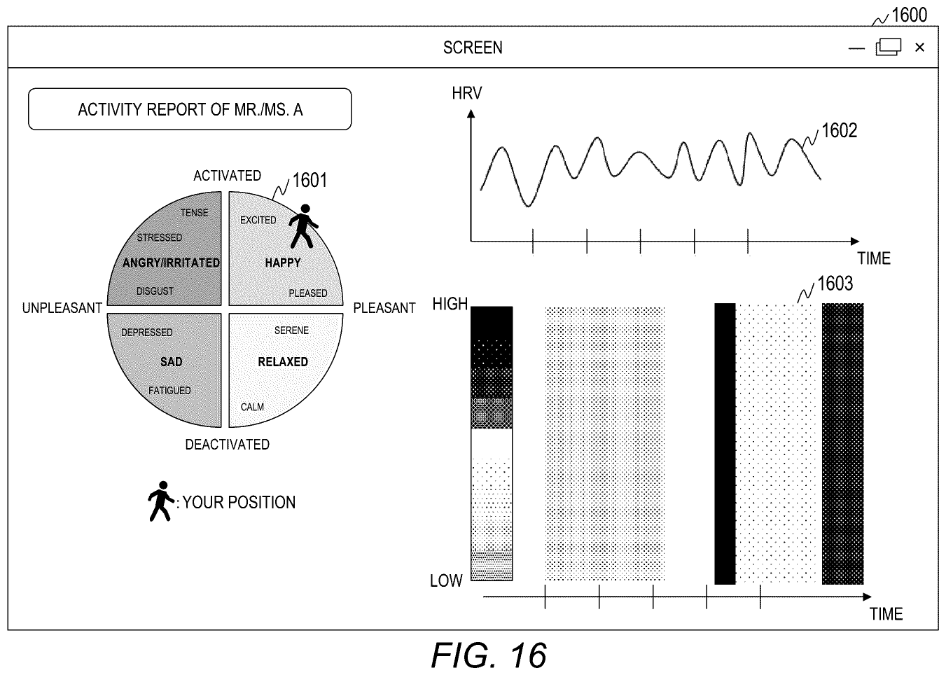

The screen presented by the display module 112 is now described. FIG. 16 is a view for illustrating an example of the screen presented by the emotion estimation system 100 in the first embodiment.

The display module 112 of the emotion estimation system 100 displays a report screen 1600 of FIG. 16 on the information terminal 102.

On the report screen 1600, an emotion map 1601, a heartbeat time series graph 1602, and a motion intensity graph 1603 are displayed.

The emotion map 1601 is an emotion model to which the estimation result of the emotion of the user is mapped. The heartbeat time series graph 1602 is a graph which indicates the heartbeat time series used for the emotion estimation. The motion intensity graph 1603 is a graph for showing a transition of the motion intensity in any period.

The emotion estimation system 100 may periodically execute the emotion estimation processing and may present the report screen 1600 of FIG. 16 each time the emotion estimation processing has been executed. At this time, information for displaying the report screen 1600 may not be recorded. Moreover, the emotion estimation system 100 may periodically execute the emotion estimation processing and may accumulate the processing results. In this case, the emotion estimation system 100 displays the report screen 1600 which presents the estimation result of the emotion at any time in response to a request from the user.

The present invention is not limited to the above embodiment and includes various modification examples. In addition, for example, the configurations of the above embodiment are described in detail so as to describe the present invention comprehensibly. The present invention is not necessarily limited to the embodiment that is provided with all of the configurations described. In addition, a part of each configuration of the embodiment may be removed, substituted, or added to other configurations.

A part or the entirety of each of the above configurations, functions, processing units, processing means, and the like may be realized by hardware, such as by designing integrated circuits therefor. In addition, the present invention can be realized by program codes of software that realizes the functions of the embodiment. In this case, a storage medium on which the program codes are recorded is provided to a computer, and a CPU that the computer is provided with reads the program codes stored on the storage medium. In this case, the program codes read from the storage medium realize the functions of the above embodiment, and the program codes and the storage medium storing the program codes constitute the present invention. Examples of such a storage medium used for supplying program codes include a flexible disk, a CD-ROM, a DVD-ROM, a hard disk, a solid state drive (SSD), an optical disc, a magneto-optical disc, a CD-R, a magnetic tape, a non-volatile memory card, and a ROM.

The program codes that realize the functions written in the present embodiment can be implemented by a wide range of programming and scripting languages such as assembler, C/C++, Perl, shell scripts, PHP, Python and Java.

It may also be possible that the program codes of the software that realizes the functions of the embodiment are stored on storing means such as a hard disk or a memory of the computer or on a storage medium such as a CD-RW or a CD-R by distributing the program codes through a network and that the CPU that the computer is provided with reads and executes the program codes stored on the storing means or on the storage medium.

In the above embodiment, only control lines and information lines that are considered as necessary for description are illustrated, and all the control lines and information lines of a product are not necessarily illustrated. All of the configurations of the embodiment may be connected to each other.

What is claimed is:

1. A computer system, comprising at least one computer,
the at least one computer including:
a processor;
a storage device coupled to the processor; and
a coupling interface coupled to the processor,
the processor being configured to:
obtain, from a user, biological data including a biological signal of the user and motion data including a motion signal relating to a motion of the user and store the biological data and the motion data in the storage device;
generate a biological signal time series and a motion signal time series through use of the biological data and the motion data in any time range;
correct, through use of the motion signal time series, the biological signal time series to a corrected biological signal time series having reduced influence of the motion of the user; and
estimate emotion of the user through use of a first biological feature amount calculated from the corrected biological signal time series, and store an emotion estimation result of the user in the storage device.

2. The computer system according to claim 1, wherein the processor is configured to correct the biological signal time series to the corrected biological signal time series through use of a motion feature amount calculated from the motion signal time series.

3. The computer system according to claim 2, wherein the processor is configured to estimate the emotion of the user through use of the motion feature amount, the corrected biological signal time series, and the first biological feature amount.

4. The computer system according to claim 3, wherein the processor is configured to:
determine whether the correction of the biological signal time series is required based on the motion feature amount; and
estimate the emotion of the user through use of the motion feature amount, the biological signal time series, and a second biological feature amount calculated from the biological signal time series in a case where the correction of the biological signal time series is not required.

5. The computer system according to claim 2,
wherein the storage device is configured to store a first model for generating the corrected biological signal time series and a second model for discriminating whether a biological signal time series input thereto is a biological signal time series at a calm time, and
wherein the processor is configured to execute learning processing including:
processing of obtaining the motion signal time series, the biological signal time series, and a ground-truth biological signal time series;
processing of inputting, to the first model, the motion feature amount calculated from the obtained motion signal time series and the first biological feature amount calculated from the obtained biological signal time series;
processing of inputting, to the second model, the obtained ground-truth biological signal time series and the corrected biological signal time series output from the first model;
processing of calculating a value of a loss function defined from output of the first model and output of the second model; and
processing of updating the first model and the second model based on the value of the loss function.

6. The computer system according to claim 2,
wherein the biological signal is a signal relating to a heartbeat, and
wherein the motion signal is a signal relating to an acceleration.

7. The computer system according to claim 2, wherein the processor is configured to present an interface for displaying an estimation result of the emotion of the user.

8. An emotion estimation method, which is executed by a computer system including at least one computer,
the at least one computer including:
a processor;
a storage device coupled to the processor; and
a coupling interface coupled to the processor,
the emotion estimation method including:
a first step of obtaining, by the processor, from a user, biological data including a biological signal of the user and motion data including a motion signal relating to a motion of the user and storing the biological data and the motion data in the storage device;
a second step of generating, by the processor, a biological signal time series and a motion signal time series through use of the biological data and the motion data in any time range;
a third step of correcting, by the processor, through use of the motion signal time series, the biological signal time series to a corrected biological signal time series having reduced influence of the motion of the user; and
a fourth step of estimating, by the processor, emotion of the user through use of a first biological feature amount calculated from the corrected biological signal time series, and storing an emotion estimation result of the user in the storage device.

9. The emotion estimation method according to claim 8, wherein the third step includes a step of correcting, by the processor, the biological signal time series to the corrected biological signal time series through use of a motion feature amount calculated from the motion signal time series.

10. The emotion estimation method according to claim 9, wherein the fourth step includes a step of estimating, by the processor, the emotion of the user through use of the motion feature amount, the corrected biological signal time series, and the first biological feature amount.

11. The emotion estimation method according to claim 10,
wherein the second step includes a step of determining, by the processor, whether the correction of the biological signal time series is required based on the motion feature amount, and
wherein the fourth step includes a step of estimating, by the processor, the emotion of the user through use of the motion feature amount, the biological signal time series, and a second biological feature amount calculated from the biological signal time series in a case where the correction of the biological signal time series is not required.

12. The emotion estimation method according to claim 9,
wherein the storage device is configured to store a first model for generating the corrected biological signal time series and a second model for discriminating whether a biological signal time series input thereto is a biological signal time series at a calm time, and
wherein the emotion estimation method further includes a step of executing, by the processor, learning processing including:
processing of obtaining the motion signal time series, the biological signal time series, and a ground-truth biological signal time series;
processing of inputting, to the first model, the motion feature amount calculated from the obtained motion signal time series and the first biological feature amount calculated from the obtained biological signal time series;
processing of inputting, to the second model, the obtained ground-truth biological signal time series and the corrected biological signal time series output from the first model;
processing of calculating a value of a loss function defined from output of the first model and output of the second model; and
processing of updating the first model and the second model based on the value of the loss function.

13. The emotion estimation method according to claim 9,
wherein the biological signal is a signal relating to a heartbeat, and
wherein the motion signal is a signal relating to an acceleration.

14. The emotion estimation method according to claim 9, further including a step of presenting, by the processor, an interface for displaying an estimation result of the emotion of the user.
